(19) United States
(12) Patent Application Publication
(10) Pub. No.: US 2017/0216098 A1
(43) Pub. Date: Aug. 3, 2017

(54) SNOW GOGGLES ALLOWING DISASSEMBLY

(71) Applicants: HUEI LUNG LI, TAINAN CITY (TW); BORRION ENTERPRISE CO., LTD., Tainan City (TW)

(72) Inventor: HUEI LUNG LI, TAINAN CITY (TW)

(21) Appl. No.: 15/009,945

(22) Filed: Jan. 29, 2016

(51) Int. Cl. A61F 9/02

(52) U.S. Cl. CPC A61F 9/029; A61F 9/025; A61F 9/026; A61F 9/027

(57) ABSTRACT

Snow goggles allowing disassembly include a goggle frame, a lens set, a protective pad, and a strap, each rapidly detachable for replacement or cleaning. The goggle frame has a first coupling side with first and second engaging portions, a second coupling side with a third engaging portion, and a peripheral portion with two first fastening units. The lens set includes first and second lenses, a connecting member, and a lens mount with fourth and fifth engaging portions for engagement with the goggle frame. The protective pad has a pad mount with a sixth engaging portion for engagement with the goggle frame. The strap has two ends each with a second fastening unit for engagement with the corresponding first fastening unit.

### FIELD OF THE INVENTION

The present invention relates to a snow goggle structure whose goggle frame, lens set, protective pad, and strap can be detached at will.

### BACKGROUND OF THE INVENTION

Snow goggles are generally worn for skiing or other sports activities to protect the eyes from injury attributable to the sun. Typically, a pair of snow goggles includes a lens and a goggle frame, the two of which are often fixedly connected together and are not individually detachable. Lens replacement is therefore impossible if the lens breaks, let alone to suit one's preference in the style or type of the lens. As such, the conventional snow goggles leave room for improvement in practical use.

Taiwan Utility Model No. M434956, published on Aug. 1, 2012 and titled “SNOW GOGGLE STRUCTURE”, discloses snow goggles including: a main frame having a peripheral portion, wherein the peripheral portion has an upper edge, a lower edge, and two lateral edges and is provided with a plurality of engaging holes, and wherein the main frame has a lateral side provided with a through hole corresponding to a side of the engaging hole in one of the lateral edges; and a lens frame having a front end coupled with an integrally formed lens and a rear end having a peripheral portion, wherein the peripheral portion has an upper edge, a lower edge, and two lateral edges and is provided with fastening members corresponding to and engaged with the engaging holes in the main frame respectively. In addition, each of the two lateral edges of the main frame is provided with a positioning hole, and each of the two lateral edges of the lens frame is provided with a positioning block corresponding to and engaged in one of the positioning holes. With the fastening members respectively engaged in the engaging holes, the lens frame is securely connected to the main frame.

While the snow goggles in the afore-cited patent allow lens replacement, their strap cannot be replaced as needed with a strap of a different style or color. Moreover, the lens frame of such snow goggles is generally provided with a foam-made protective pad on the side adjacent to the user's face to ensure a tight fit between the lens frame and the face and to increase comfort. This foam-made protective pad, however, is not detachable for cleaning or replacement and hence leaves something to be desired.

### SUMMARY OF THE INVENTION

In view of the aforesaid shortcomings of the existing snow goggles, the present invention provides snow goggles which can be disassembled. The snow goggles of the present invention include a goggle frame, a lens set, a protective pad, and a strap. The goggle frame is provided with a first coupling side and a second coupling side facing away from the first coupling side. Formed between the first coupling side and the second coupling side is a peripheral portion provided with two first fastening units. The first coupling side is provided with at least one first engaging portion and at least one second engaging portion. The second coupling side is provided with at least one third engaging portion. The lens set is securely connected to the first coupling side of the goggle frame in a detachable manner and includes a first lens, a second lens, a connecting member, and a lens mount. The connecting member is secured between the first lens and the second lens and forms a furrow between the first lens and the second lens. The lens mount is configured to be securely engaged in the furrow and is provided with a fourth engaging portion and a fifth engaging portion, which correspond to the first engaging portion and the second engaging portion of the goggle frame respectively. The fourth engaging portion is configured to be securely engaged with the first engaging portion while the fifth engaging portion is configured to be securely engaged with the second engaging portion. The protective pad is securely connected to the second coupling side of the goggle frame in a detachable manner and is provided with a pad mount. The pad mount is provided with a sixth engaging portion corresponding to the third engaging portion of the goggle frame. The sixth engaging portion is securely engaged with the third engaging portion. The strap is fastened to the goggle frame in a detachable manner and has two ends each provided with a second fastening unit corresponding to one of the first fastening units of the goggle frame. Each second fastening unit is securely engaged with a corresponding one of the first fastening units.

Preferably, the first engaging portion is an engaging groove, and the fourth engaging portion is an engaging block configured to be securely engaged in the engaging groove.

Preferably, the second engaging portion is an engaging groove, and the fifth engaging portion is an engaging block configured to be securely engaged in the engaging groove.

Preferably, the third engaging portion is an engaging groove, and the sixth engaging portion is an engaging block configured to be securely engaged in the engaging groove.

Preferably, the first engaging portion is protrudingly provided therein with a positioning pin, and the fourth engaging portion is provided with a pin hole corresponding to the positioning pin. The positioning pin is configured to be engaged and positioned in the pin hole.

Preferably, the two first fastening units are provided at two lateral sides of the peripheral portion respectively. Each first fastening unit is provided therein with a fastening groove, has an external portion with an upper end and a lower end each provided with an opening penetrating through the fastening groove, and is further provided with two pressing portions corresponding to the openings respectively. Each second fastening unit has an upper end and a lower end each provided with an elastic hook. The second fastening units are configured to be engaged in the fastening grooves respectively, with the two elastic hooks of each second fastening unit engaged in the two openings of the corresponding one of the first fastening units respectively.

The technical features described above have the following advantages:

1. The lens set can be rapidly detached from the goggle frame in order to be replaced with a lens set of a different style or type. This allows the user to choose the lens set according to weather, temperature, or the sports activity to partake in.

2. The protective pad can be rapidly detached from the goggle frame in order to be replaced or cleaned. This solves hygiene issues associated with sweating during a sports activity, for the protective pad may turn into a hotbed for bacteria when wetted or made dirty by sweat.

3. The strap can be rapidly detached from the goggle frame in order to be replaced with a strap of a different style or color. This allows the user to change the strap as required by the sports activity to take part in, or according to personal preference, or when the strap is dirty.

### BRIEF DESCRIPTION OF THE DRAWINGS

FIG. 1 is an exploded perspective view of an embodiment of the present invention;

FIG. 2 is an assembled perspective view of an embodiment of the present invention;

FIG. 3 is a perspective view of the goggle frame in an embodiment of the present invention;

FIG. 4 is another perspective view of the goggle frame in FIG. 3, as viewed from a different angle;

FIG. 5 is an exploded perspective view of the lens set in an embodiment of the present invention;

FIG. 6 is an assembled sectional view of the lens set in an embodiment of the present invention;

FIG. 7 is a partial perspective view of the goggle frame and the lens set in an embodiment of the present invention;

FIG. 8 is a partial assembled perspective view of the goggle frame and the lens set in FIG. 8;

FIG. 9 is an enlarged perspective view of the second engaging portion and the fifth engaging portion in an embodiment of the present invention;

FIG. 10 is an enlarged assembled perspective view of the second engaging portion and the fifth engaging portion in FIG. 9;

FIG. 11 is a partial perspective view of the goggle frame and the protective pad in an embodiment of the present invention;

FIG. 12 is a partial assembled perspective view of the goggle frame and the protective pad in FIG.11; and

FIG. 13 is a partial assembled perspective view of the goggle frame and the strap in an embodiment of the present invention.

### DETAILED DESCRIPTION OF THE INVENTION

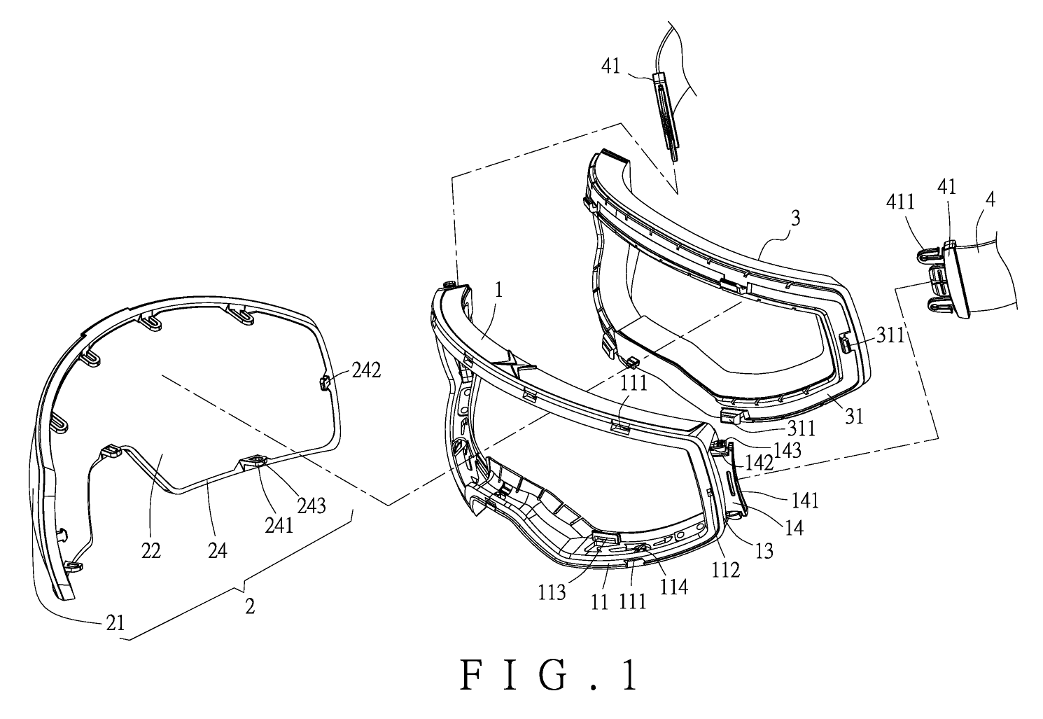

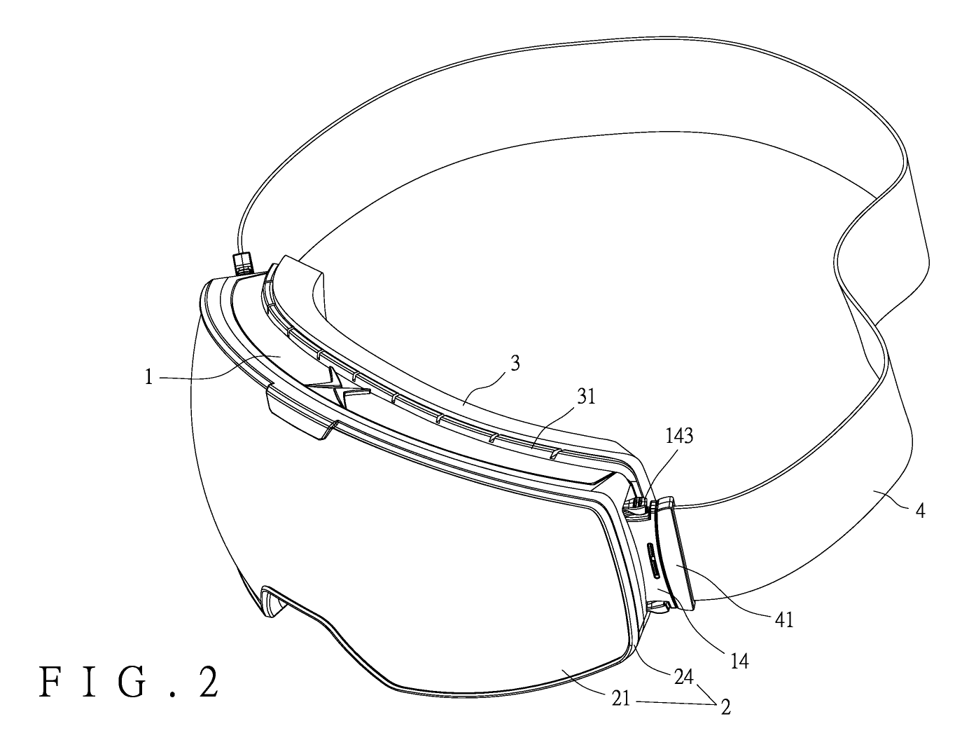

Referring to FIG. 1 and FIG. 2, an embodiment of the present invention includes a goggle frame 1, a lens set 2, a protective pad 3, and a strap 4.

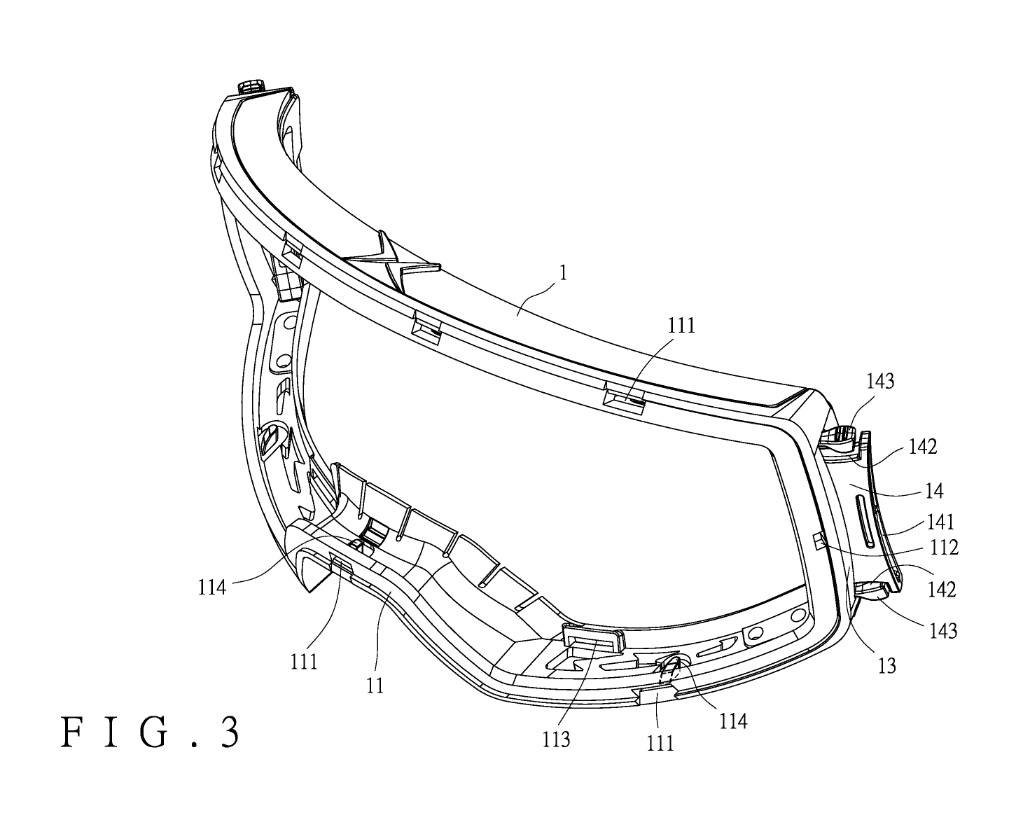

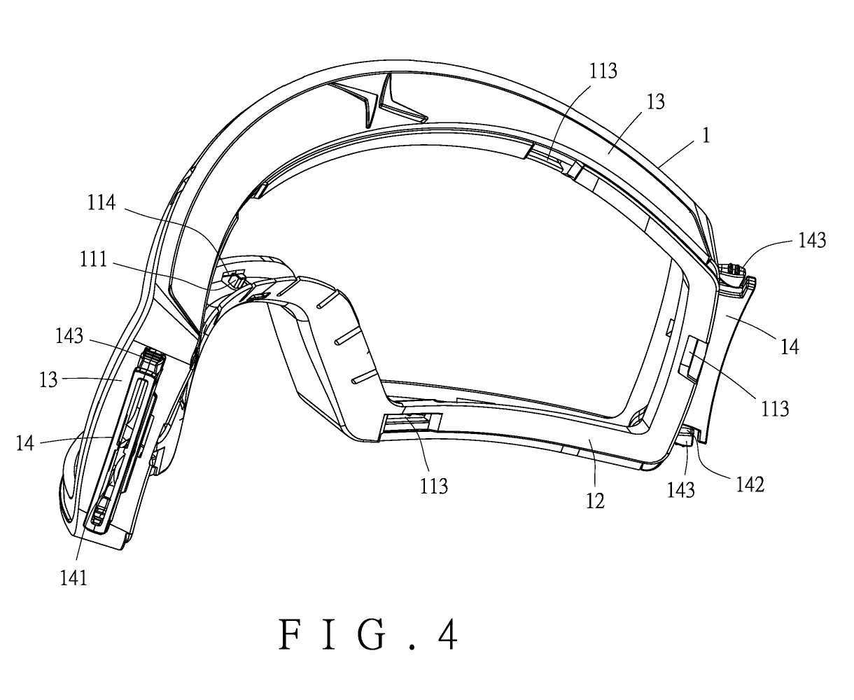

As shown in FIG. 3 and FIG. 4, the goggle frame 1 is provided with a first coupling side 11 at the front and a second coupling side 12 at the rear such that the two coupling sides face away from each other. A peripheral portion 13 is provided between, and along the peripheries of, the first coupling side 11 and the second coupling side 12. The peripheral portion 13 has two lateral sides each provided with a first fastening unit 14. The first coupling side 11 is provided with at least one first engaging portion 111 and at least one second engaging portion 112. The second coupling side 12 is provided with at least one third engaging portion 113.

The first engaging portion 111, the second engaging portion 112, and the third engaging portion 113 are engaging grooves, and the first engaging portion 111 is protrudingly provided therein with a positioning pin 114. In addition, each first fastening unit 14 is provided therein with a fastening groove 141. The upper as well as the lower end of an external portion of each first fastening unit 14 is provided with an opening 142 penetrating through the fastening groove 141. Each first fastening unit 14 is further provided with two pressing portions 143 corresponding respectively to the openings 142.

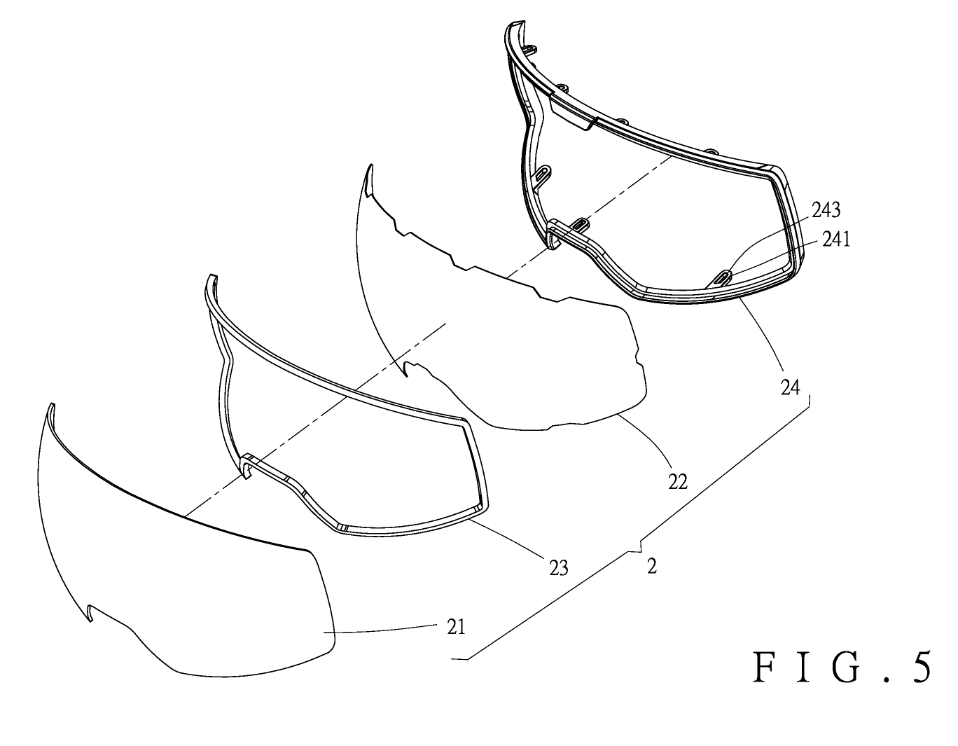

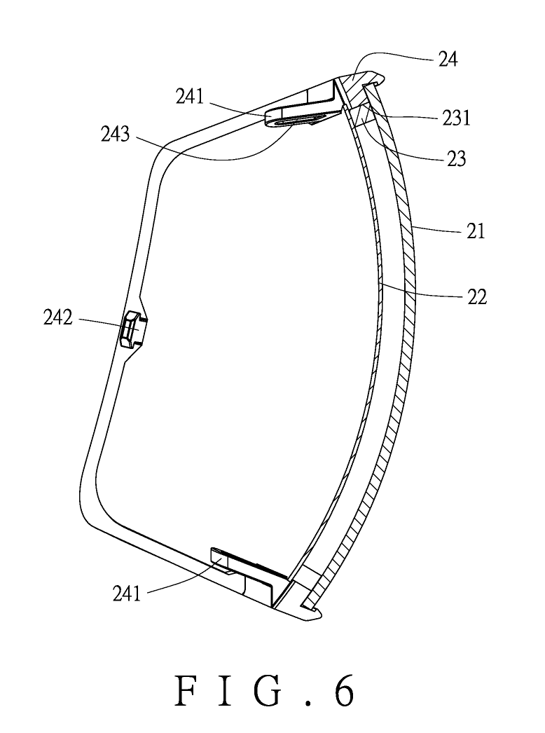

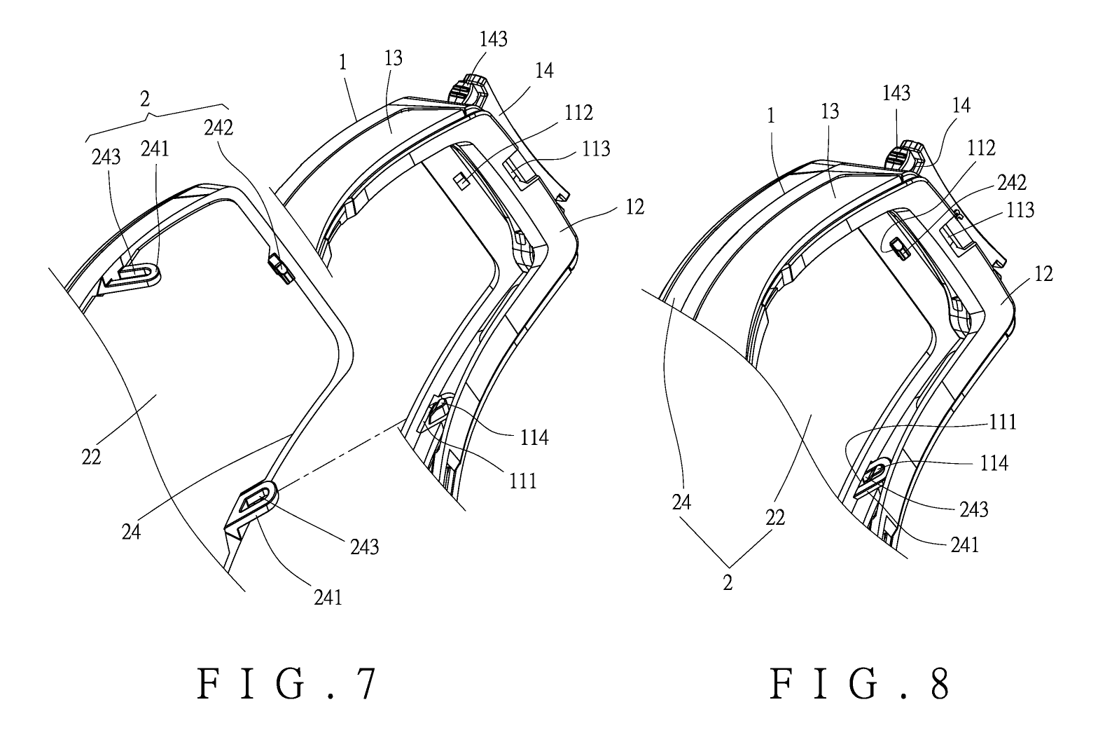

As shown in FIG. 5 and FIG. 6, the lens set 2 is securely connected to the first coupling side 11 of the goggle frame 1 in a detachable manner. The lens set 2 includes a first lens 21, a second lens 22, a connecting member 23, and a lens mount 24. The connecting member 23 is adhesively secured between the first lens 21 and the second lens 22. The connecting member 23 has an outer periphery located between the first lens 21 and the second lens 22 and formed with a furrow 231. The lens mount 24 is engaged and secured in the furrow 231 such that the first lens 21 and the second lens 22 are connected and are secured opposite each other. The lens mount 24 is provided with a fourth engaging portion 241 and a fifth engaging portion 242 which correspond respectively to the first engaging portion 111 and the second engaging portion 112 of the goggle frame 1. The fourth engaging portion 241 and the fifth engaging portion 242 are engaging blocks. Furthermore, the fourth engaging portion 241 is provided with a pin hole 243 corresponding to the positioning pin 114. As shown in FIG. 7 and FIG. 8, the fourth engaging portion 241 is securely engaged in the first engaging portion 111, with the positioning pin 114 of the first engaging portion 111 engaged and positioned in the pin hole 243 of the fourth engaging portion 241. The fifth engaging portion 242, on the other hand, is securely engaged in the second engaging portion 112, as shown in

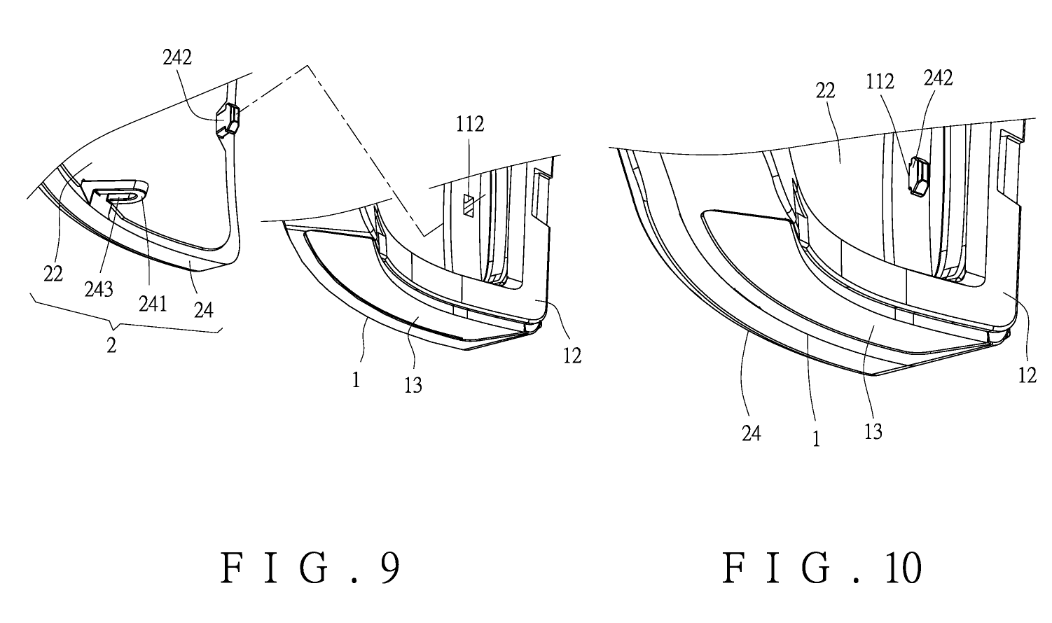

FIG. 9 and FIG. 10. Thus, the lens set 2 is secured to the first coupling side 11 of the goggle frame 1.

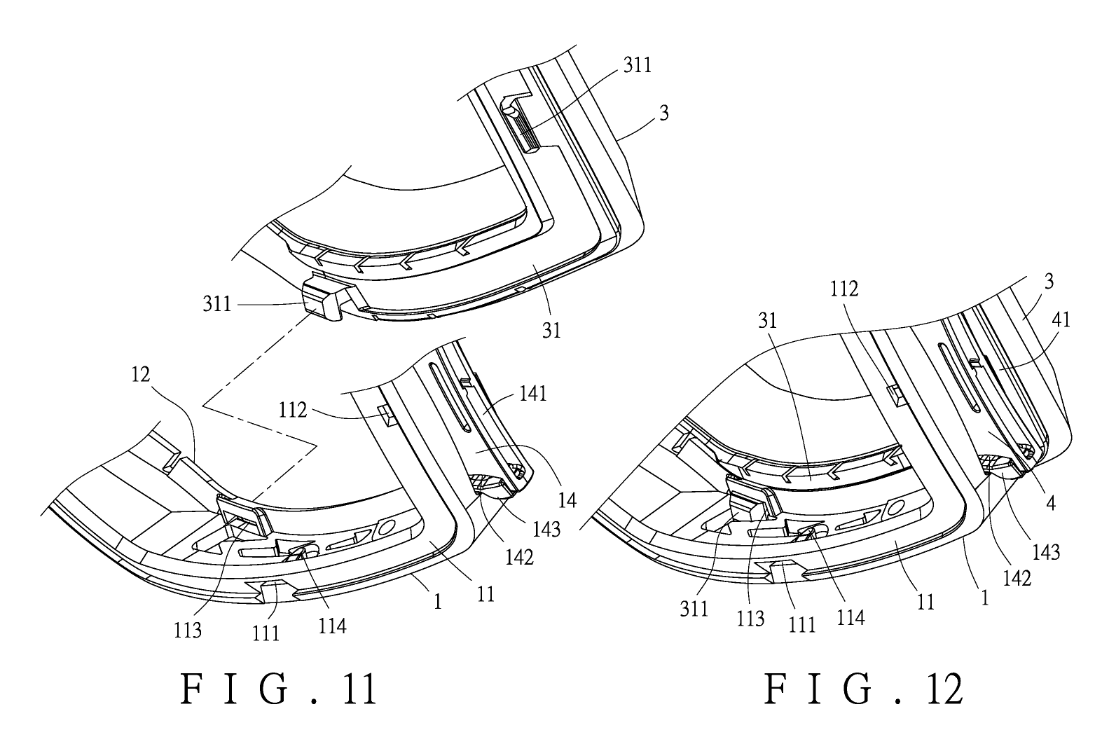

Referring to FIG. 1, FIG. 11, and FIG. 12, the protective pad 3 is securely connected to the second coupling side 12 of the goggle frame 1 in a detachable manner. The protective pad 3 is provided with a pad mount 31. The pad mount 31 is provided with a sixth engaging portion 311 corresponding to the third engaging portion 113 of the goggle frame 1. The sixth engaging portion 311 is an engaging block and is securely engaged in the third engaging portion 113 to secure the protective pad 3 to the second coupling side 12 of the goggle frame 1. The protective pad 3 is made of an elastic foam material.

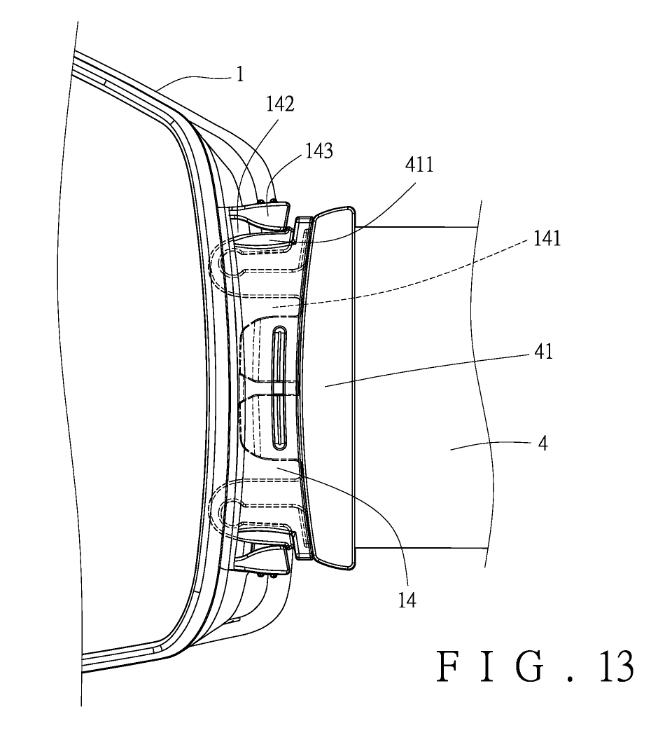

Referring to FIG. 1 and FIG. 13, the strap 4 is fastened to the goggle frame 1 in a detachable manner. Each of the two ends of the strap 4 is provided with a second fastening unit 41 corresponding to one of the first fastening units 14 of the goggle frame 1. The upper as well as the lower end of each second fastening unit 41 is provided with an elastic hook 411. The second fastening units 41 are configured to be engaged in the fastening grooves 141 of the first fastening units 14 respectively, with the two elastic hooks 411 of each second fastening unit 41 respectively engaged in the two openings 142 of the corresponding first fastening unit 14, thereby fastening the strap 4 to the two lateral sides of the peripheral portion 13 of the goggle frame 1.

Referring to FIG. 1, FIG. 7, and FIG. 8, when it is desired to detach the lens set 2 and replace it with a lens set 2 of a different style or type to adapt to a certain weather, temperature, or sports activity, a hand tool is used to disengage the fourth engaging portion 241 and the fifth engaging portion 242 of the lens mount 24 from the first engaging portion 111 and the second engaging portion 112 respectively. Then, with the positioning pin 114 of the first engaging portion 112 disengaged from the pin hole 243 of the fourth engaging portion 241, the fourth engaging portion 241 is separated from the first engaging portion 111, and, as shown in FIG. 9 and FIG. 10, the fifth engaging portion 242 is separated from the second engaging portion 112. The lens set 2 can now be detached from the first coupling side 11 of the goggle frame 1 and rapidly replaced with a different lens set 2.

Referring to FIG. 1, FIG. 11, and FIG. 12, when it is desired to detach the protective pad 3 for cleaning or replacement, the sixth engaging portion 311 of the pad mount 31 is disengaged from the third engaging portion 113 of the goggle frame 1 with a hand tool and then separated from the third engaging portion 113 of the goggle frame 1, allowing the protective pad 3 to be detached from the second coupling side 12 of the goggle frame 1 and either rapidly replaced or cleaned. This helps maintain hygiene and prevent bacterial growth which tends to occur when the protective pad is wetted or made dirty by sweat during a sports activity.

Referring to FIG. 1 and FIG. 13, when it is desired to detach the strap 4 for cleaning or replacement, either due to the user's needs or preference or when the strap 4 is dirty after long-term use, the two pressing portions 143 of each first fastening unit 14 can be pressed singlehandedly in order for the two pressing portions 143 to press the two elastic hooks 411 of the corresponding second fastening unit 41 respectively. Once the two elastic hooks 411 are elastically deformed and no longer engaged in the two openings 142 of the first fastening unit 14, the second fastening unit 41 can be pulled outward to separate from the first fastening unit 14. Thus, the strap 4 is detached from the peripheral portion 13 of the goggle frame 1 and can be rapidly replaced or cleaned.

By the foregoing operations, each of the lens set 2, the protective pad 3, and the strap 4 can be easily and rapidly detached in order to be replaced or cleaned.

The embodiment described above is but one embodiment of the present invention and is not intended to be restrictive of the scope of patent protection sought by the applicant. All simple, equivalent changes and substitutions made according to the appended claims and the disclosure of this specification should fall within the scope of the present invention.

What is claimed is:

1. Snow goggles allowing disassembly, comprising:
a goggle frame provided with a first coupling side and a second coupling side facing away from the first coupling side, there being a peripheral portion formed between the first coupling side and the second coupling side, the peripheral portion being provided with two first fastening units, the first coupling side being provided with at least one first engaging portion and at least one second engaging portion, the second coupling side being provided with at least one third engaging portion;
a lens set securely connected to the first coupling side of the goggle frame in a detachable manner, the lens set including a first lens, a second lens, a connecting member, and a lens mount, the connecting member being secured between the first lens and the second lens, the connecting member forming a furrow between the first lens and the second lens, the lens mount being configured to be securely engaged in the furrow, the lens mount being provided with a fourth engaging portion corresponding to the first engaging portion of the goggle frame and a fifth engaging portion corresponding to the second engaging portion of the goggle frame, the fourth engaging portion being configured to be securely engaged with the first engaging portion, the fifth engaging portion being configured to be securely engaged with the second engaging portion;
a protective pad securely connected to the second coupling side of the goggle frame in a detachable manner, the protective pad being provided with a pad mount, the pad mount being provided with a sixth engaging portion corresponding to the third engaging portion of the goggle frame, the sixth engaging portion being securely engaged with the third engaging portion;
and a strap fastened to the goggle frame in a detachable manner, the strap having two ends each provided with a second fastening unit corresponding to one of the first fastening units of the goggle frame, each said second fastening unit being configured to be securely engaged with a corresponding one of the first fastening units.

2. The snow goggles of claim 1, wherein the first engaging portion is an engaging groove, the fourth engaging portion is an engaging block, and the engaging block is configured to be securely engaged in the engaging groove.

3. The snow goggles of claim 1, wherein the second engaging portion is an engaging groove, the fifth engaging portion is an engaging block, and the engaging block is configured to be securely engaged in the engaging groove.

4. The snow goggles of claim 1, wherein the third engaging portion is an engaging groove, the sixth engaging portion is an engaging block, and the engaging block is configured to be securely engaged in the engaging groove.

5. The snow goggles of claim 1, wherein the first engaging portion is protrudingly provided therein with a positioning pin, the fourth engaging portion is provided with a pin hole corresponding to the positioning pin, and the positioning pin is configured to be engaged and positioned in the pin hole.

6. The snow goggles of claim 1, wherein the two first fastening units are provided at two lateral sides of the peripheral portion respectively, each said first fastening unit is provided therein with a fastening groove, each said first fastening unit has an external portion with an upper end and a lower end each provided with an opening penetrating through the fastening groove of the each said first fastening unit, each said first fastening unit is provided with two pressing portions corresponding to the openings of the each said first fastening unit respectively, each said second fastening unit has an upper end and a lower end each provided with an elastic hook, and the second fastening units are configured to be engaged in the fastening grooves respectively, with the two elastic hooks of each said second fastening unit engaged in the two openings of a corresponding one of the first fastening units respectively.

7. Snow goggles allowing disassembly, comprising:
a goggle frame provided with two first fastening units;
a lens set mounted to the goggle frame;
a protective pad attached to the goggle frame; and
a strap fastened to the goggle frame in a detachable manner, the strap having two ends each provided with a second fastening unit corresponding to one of the first fastening units of the goggle frame, each said second fastening unit being configured to be securely engaged with a corresponding one of the first fastening units.

8. The snow goggles of claim 7, wherein the goggle frame has a peripheral portion, the two second fastening units are provided at two lateral sides of the peripheral portion respectively, each said first fastening unit is provided therein with a fastening groove, each said first fastening unit has an external portion with an upper end and a lower end each provided with an opening penetrating through the fastening groove of the each said first fastening unit, each said first fastening unit is provided with two pressing portions corresponding to the openings of the each said first fastening unit respectively, each said second fastening unit has an upper end and a lower end each provided with an elastic hook, and the second fastening units are configured to be engaged in the fastening grooves respectively, with the two elastic hooks of each said second fastening unit engaged in the two openings of a corresponding one of the first fastening units respectively.
